SEQUENCE LISTING (2 sequences)

SEQ ID NO: 1 - protein, 502 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPQAQSKIEELEQEPEPEPEPEPEPEPEPEPEQL

SEQ ID NO: 2 - protein, 30 aa, Homo sapiens
GPPAAAPGHPLAPGPHPAAPSSWGPRPRRY

(19) United States
(12) Patent Application Publication
(10) Pub. No.: US 2025/0127856 A1
(43) Pub. Date: Apr. 24, 2025

(54) COMBINED PREPARATIONS FOR THE TREATMENT OF CANCER

(71) Applicant: IMMUTEP S.A.S., Saint-Aubin (FR)

(72) Inventor: Frederic Triebel, Versailles (FR)

(73) Assignee: IMMUTEP S.A.S., Saint-Aubin (FR)

(21) Appl. No.: 19/000,820

(22) Filed: Dec. 24, 2024

Related U.S. Application Data
(63) Continuation of application No. 16/918,527, filed on Jul. 1, 2020, now Pat. No. 12,214,012.
Application No. 16/918,527 is a continuation of application No. 15/105,789, filed on Jun. 17, 2016, now Pat. No. 10,736,940.

(30) Foreign Application Priority Data
Dec. 19, 2013 (GB) 1322626.1

(51) Int. Cl. A61K 38/17; A61K 31/282; A61K 31/4745; A61K 31/555; A61K 45/06; C07K 14/705

(52) U.S. Cl. CPC A61K 38/1774; A61K 31/282; A61K 31/4745; A61K 31/555; A61K 45/06; C07K 14/70503; C07K 2319/30

(57) ABSTRACT

Combined preparations for the treatment of cancer are described. The combined preparations comprise: (a) LAG-3 protein, or a derivative thereof that is able to bind to MHC class II molecules; and (b) an anti-neoplastic agent, wherein the anti-neoplastic agent is a platinum-based anti-neoplastic agent or a topoisomerase I inhibitor. Methods for the treatment of cancer using the combined preparations are also described.

### CROSS REFERENCE TO RELATED APPLICATIONS

This application is a continuation of U.S. application Ser. No. 16/918,527, filed Jul. 1, 2020, which is a continuation of U.S. application Ser. No. 15/105,789, filed Jun. 17, 2016, now U.S. Pat. No. 10,736,940, which is a National Stage Entry of International Application No. PCT/EP2014/078779, filed Dec. 19, 2014, which claims priority from GB 1322626.1, filed on Dec. 19, 2013, the entire contents of which are incorporated herein by reference herein.

### INCORPORATION BY REFERENCE OF SEQUENCE LISTING

The Sequence Listing in the XML file, named as 33684ZY_SequenceListing.xml of 4,096 bytes, created on Dec. 23, 2024 and submitted to the United States Patent and Trademark Office via Patent Center, is incorporated herein by reference.

This invention relates to combined preparations and to pharmaceutical compositions, and their use as medicaments, in particular for the treatment of cancer, and to methods for the treatment of cancer.

Cancer may be treated with one or more cytotoxic anti-neoplastic drugs (“chemotherapeutic agents”) as part of a standardized regimen. Chemotherapy may be aimed at curing a patient, or at prolonging life, or alleviating symptoms.

Conventional chemotherapeutic agents act by killing cells that divide rapidly, exploiting one of the properties of most cancer cells. However, chemotherapy also harms cells that divide rapidly under normal circumstances, for example cells in the bone marrow, digestive tract, and hair follicles. This causes the most common side-effects of chemotherapy: myelosuppression (decreased production of blood cells, hence also immunosuppression), mucositis (inflammation of the lining of the digestive tract), and alopecia (hair loss).

There is a need to provide more effective cancer treatments, and to provide effective cancer treatments with reduced side effects.

The lymphocyte activation gene 3 (LAG-3) is a CD4 homolog type I membrane protein with four extracellular Ig superfamily domains. Similar to CD4, LAG-3 oligomerizes at the surfaces of T cells and binds to MHC class II molecules on antigen-presenting cells (APCs) but with significantly higher affinity than CD4. LAG-3 is expressed on activated CD4-positive and CD8-positive T lymphocytes where it associates with the CD3-TCR complex at the cell surface and negatively regulates signal transduction. As a consequence, it negatively regulates T cell proliferation, function, and homeostasis.

LAG-3-derived soluble fusion proteins, have been shown to bind MHC class Il molecules with a much higher avidity than CD4, to increase the capacity of MHC class Il-positive macrophages and immature dendritic cells to induce T cell responses in vitro, and to enhance the in vitro induction of viral and tumor-specific cytotoxic T cells. Accordingly, a LAG-3 fusion protein is used as a systemic immunostimulant and as an adjuvant for cancer vaccines.

WO 2009/044273 describes use of recombinant LAG-3 protein, or derivatives thereof, for boosting a monocyte-mediated immune response, in particular to induce an increase in the number of monocytes in blood, for the treatment of cancer.

It has now surprisingly been found that administration of LAG-3 protein, or a derivative thereof that is able to bind to MHC class Il molecules, and a platinum-based anti-neoplastic agent, or a topoisomerase I inhibitor, has a synergistic effect on reducing tumor growth.

According to the invention there is provided a combined preparation, which comprises: (a) LAG-3 protein, or a derivative thereof that is able to bind to MHC class Il molecules; and (b) an anti-neoplastic agent, wherein the anti-neoplastic agent is a platinum-based anti-neoplastic agent or a topoisomerase I inhibitor.

The term “combined preparation” as used herein refers to a “kit of parts” in the sense that the combination components (a) and (b) as defined above can be dosed independently or by use of different fixed combinations with distinguished amounts of the combination components (a) and (b). The components can be administered simultaneously or one after the other. If the components are administered one after the other, preferably the time interval between administration is chosen such that the therapeutic effect of the combined use of the components is greater than the effect which would be obtained by use of only any one of the combination components (a) and (b).

The components of the combined preparation may be present in one combined unit dosage form, or as a first unit dosage form of component (a) and a separate, second unit dosage form of component (b). The ratio of the total amounts of the combination component (a) to the combination component (b) to be administered in the combined preparation can be varied, for example in order to cope with the needs of a patient sub-population to be treated, or the needs of the single patient, which can be due, for example, to the particular disease, age, sex, or body weight of the patient.

Preferably, there is at least one beneficial effect, for example an enhancing of the effect of the anti-neoplastic agent, or a mutual enhancing of the effect of the combination components (a) and (b), for example a more than additive effect, additional advantageous effects, fewer side effects, less toxicity, or a combined therapeutic effect compared with an effective dosage of one or both of the combination components (a) and (b), and very preferably a synergism of the combination components (a) and (b).

A combined preparation of the invention may be provided as a pharmaceutical combined preparation for administration to a mammal, preferably a human. The LAG-3 protein, or derivative thereof, may optionally be provided together with a pharmaceutically acceptable carrier, excipient, or diluent, and/or the anti-neoplastic agent may optionally be provided together with a pharmaceutically acceptable carrier, excipient, or diluent.

The LAG-3, or derivative thereof, may be present at a dose which is a molar equivalent of 0.25-30 mg, 1-30 mg, or 6-30 mg of the LAG-3 derivative LAG-3Ig fusion protein IMP321. Doses of 6-30 mg per subcutaneous (s.c.) injection of IMP321 have been shown to be safe and provide an acceptable systemic exposure based on the results of pharmacokinetics data obtained in metastatic renal cell cancer patients. A blood concentration of IMP321 superior to 1 ng/ml for at least 24 hours after s.c. injection is obtained in patients injected with IMP321 doses of more than 6 mg.

A combined preparation of the invention may comprise a plurality of doses of the LAG-3 protein, or derivative thereof.

The dose of the anti-neoplastic agent will depend on the particular anti-neoplastic agent being used.

Platinum-based anti-neoplastic agents are coordination complexes of platinum used in cancer chemotherapy. They are believed to form cross-links in DNA that inhibit DNA repair and/or DNA synthesis resulting in cell death. The main dose-limiting side effect of cancer treatment using platinum compounds is peripheral neurotoxicity. Examples of platinum-based anti-neoplastic agents include cisplatin, carboplatin, oxaliplatin, satraplatin, picoplatin, Nedaplatin, and Triplatin. Carboplatin, or cis-diammine(cyclobutane-1,1-dicarboxylate-O,O′)platinum(II) (trade names Paraplatin and Paraplatin-AQ), is used against some forms of cancer (mainly ovarian carcinoma, lung, head and neck cancers as well as endometrial, esophageal, bladder, breast and cervical; central nervous system or germ cell tumors; osteogenic sarcoma, and as preparation for a stem cell or bone marrow transplant). It has greatly reduced side-effects compared to its parent compound cisplatin. Guidelines for carboplatin dosing are available from the US Food and Drug Administration (FDA).

Oxaliplatin, or [(1R,2R)-cyclohexane-1,2-diamine](ethanedioato-O,O′)platinum(II) (trade name Eloxatin), comprises a square planar platinum(II) centre. In contrast to cisplatin and carboplatin, oxaliplatin comprises the bidentate ligand 1,2-diaminocyclohexane in place of the two monodentate ammine ligands. It also has a bidentate oxalate group. Oxaliplatin has anti-tumor activity against colon carcinoma. Oxaliplatin functions by forming both inter- and intra-strand cross links in DNA. Cross links in DNA prevent DNA replication and transcription, resulting in cell death. The recommended dose of oxaliplatin in an adjuvant setting is 85 mg/m² intravenously repeated every two weeks for 12 cycles. A recommended dose for oxaliplatin in treatment of metastatic colorectal cancer is 85 mg/m² intravenously repeated every two weeks until disease progression or unacceptable toxicity.

Topoisomerase inhibitors are agents designed to interfere with the action of topoisomerase enzymes (topoisomerase I and II), which are enzymes that control changes in DNA structure by catalyzing the breaking and rejoining of the phosphodiester backbone of DNA strands during the normal cell cycle. It is thought that topoisomerase inhibitors block the ligation step of the cell cycle, generating single and double stranded breaks that harm the integrity of the genome. Introduction of these breaks subsequently leads to apoptosis and cell death.

Human DNA topoisomerase I (Top1) is an essential enzyme that relaxes DNA supercoiling during replication and transcription. Top1 generates DNA single-strand breaks that allow rotation of the cleaved strand around the double helix axis. Top1 also re-ligates the cleaved strand to re-establish intact duplex DNA. Top1-DNA intermediates, known as cleavage complexes, are transient and present at low levels under normal circumstances. However, treatment with Top1 inhibitors, such as the camptothecins, stabilizes the cleavable complexes, prevents DNA religation and induces lethal DNA strand breaks. Cancer cells are selectively sensitive to the generation of these DNA lesions.

Topotecan, or (S)-10-[(dimethylamino)methyl]-4-ethyl-4,9-dihydroxy-1H-pyrano[3′,4′:6,7]indolizino [1,2-b] quinoline-3,14(4H,12H)-dione monohydrochloride (trade name Hycamtin), is a chemotherapeutic agent that is a topoisomerase I inhibitor. It is a water-soluble derivative of camptothecin. It is used in form of the hydrochloride to treat ovarian cancer and lung cancer, as well as other cancer types. Topotecan is a semi-synthetic derivative of camptothecin. Camptothecin is a natural product extracted from the bark of the tree Camptotheca acuminata. Topoisomerase-I is a nuclear enzyme that relieves torsional strain in DNA by opening single strand breaks. Once topoisomerase-I creates a single strand break, the DNA can rotate in front of the advancing replication fork. Topotecan intercalates between DNA bases. This intercalation disrupts the DNA duplication machinery when it reaches a site where topotecan is intercalated. This disruption prevents DNA replication, and ultimately leads to cell death. Mammalian cells cannot efficiently repair these double strand breaks. This process leads to breaks in the DNA strand resulting in apoptosis.

A recommended dose of Hycamtin capsules is 2.3 mg/m² body surface area/day administered for five consecutive days with a three week interval between the start of each course.

Another camptothecin derivative irinotecan (CPT11) is approved for the treatment of colon cancer.

A combined preparation of the invention may comprise a plurality of doses of the anti-neoplastic agent.

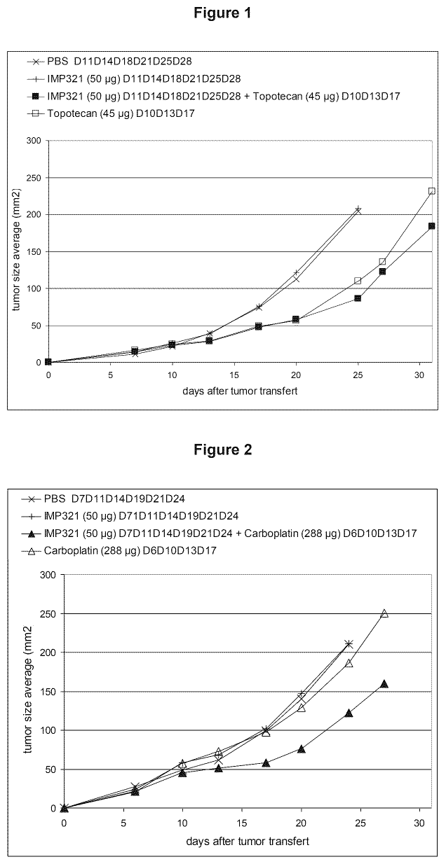

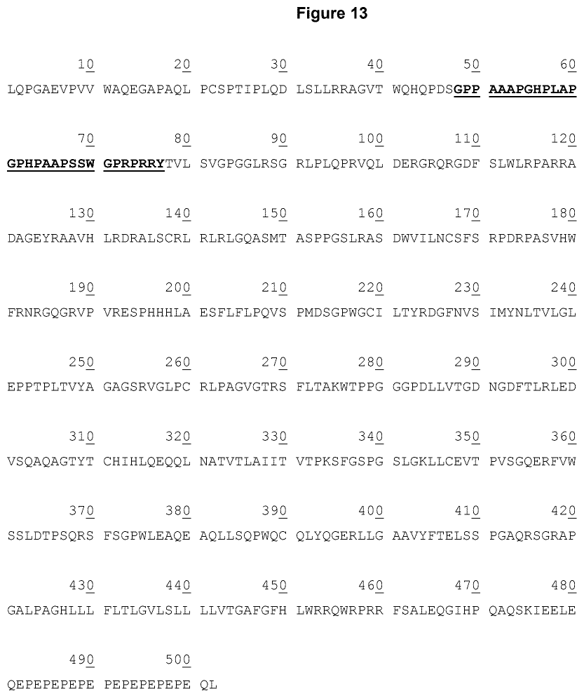

The LAG-3 protein may be an isolated natural or recombinant LAG-3 protein. The LAG-3 protein may comprise an amino sequence of LAG-3 protein from any suitable species, such as a primate or murine LAG-3 protein, but preferably a human LAG-3 protein. The amino acid sequence of human and murine LAG-3 protein is provided in FIG. 1 of Huard et al (Proc. Natl. Acad. Sci. USA, 11:5744-5749, 1997). The sequence of human LAG-3 protein is repeated in FIG. 13 below (SEQ ID NO: 1). The amino acid sequences of the four extracellular Ig superfamily domains (D1, D2, D3, and D4) of human LAG-3 are also identified in FIG. 1 of Huard et al., at amino acid residues: 1-149 (D1); 150-239 (D2); 240-330 (D3); and 331-412 (D4).

Derivatives of LAG-3 protein include fragments, variants, or mutants of LAG-3 protein that are able to bind MHC class II molecules. Several derivatives of LAG-3 protein are known that are able to bind to MHC class II molecules. Many examples of such derivatives are described in Huard et al (Proc. Natl. Acad. Sci. USA, 11:5744-5749, 1997). This document describes characterization of the MHC class II binding site on LAG-3 protein. Methods for making mutants of LAG-3 are described, as well as a quantitative cellular adhesion assay for determining the ability of LAG-3 mutants to bind class II-positive Daudi cells. Binding of several different mutants of LAG-3 to MHC class II molecules was determined. Some mutations were able to reduce class II binding, while other mutations increased the affinity of LAG-3 for class II molecules. Many of the residues essential for binding MHC class II proteins are clustered at the base of a large 30 amino acid extra-loop structure in the LAG-3 D1 domain. The amino acid sequence of the extra-loop structure of the D1 domain of human LAG-3 protein is GPPAAAPGHPLAPGPHPAAPSSWGPRPRRY (SEQ ID NO: 2), the underlined sequence in FIG. 13.

The LAG-3 protein derivative may comprise the 30 amino acid extra-loop sequence of the human LAG-3 D1 domain, or a variant of such sequence with one or more conservative amino acid substitutions. The variant may comprise amino acid sequence that has at least 70%, 80%, 90%, or 95% amino acid identity with the 30 amino acid extra-loop sequence of the human LAG-3 D1 domain.

The derivative of LAG-3 protein may comprise an amino acid sequence of domain D1, and optionally domain D2, of LAG-3 protein, preferably human LAG-3 protein.

The derivative of LAG-3 protein may comprise an amino acid sequence that has at least 70%, 80%, 90%, or 95% amino acid identity with domain D1, or with domain D1 and D2, of LAG-3 protein, preferably human LAG-3 protein.

The derivative of LAG-3 protein may comprise an amino acid sequence of domains D1, D2, D3, and optionally D4, of LAG-3 protein, preferably human LAG-3 protein.

The derivative of LAG-3 protein may comprise an amino acid sequence that has at least 70%, 80%, 90%, or 95% amino acid identity with domain D1, D2, and D3, or with domain D1, D2, D3, and D4, of LAG-3 protein, preferably human LAG-3.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215:403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms

(Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

The LAG-3 protein derivative may be fused to Immunoglobulin Fc amino acid sequence, preferably human IgG1 Fc amino acid sequence, optionally by a linker amino acid sequence.

The ability of a derivative of LAG-3 protein to bind to MHC class Il molecules may be determined using a quantitative cellular adhesion assay as described in Huard et al (supra). The affinity of a derivative of LAG-3 protein for MHC class Il molecules may be at least 20%, 30%, 40%, 50%, 60%, 70%, 80%, 90%, or 100% of the affinity of human LAG-3 protein for class Il molecules. Preferably the affinity of a derivative of LAG-3 protein for MHC class Il molecules is at least 50% of the affinity of human LAG-3 protein for class II molecules.

Examples of suitable derivatives of LAG-3 protein that are able to bind MHC class II molecules include derivatives comprising:
amino acid residues 23 to 448 of the human LAG-3 sequence;
amino acid sequence of domains D1 and D2 of LAG-3;
amino acid sequence of domains D1 and D2 of LAG-3 with an amino acid substitution at one or more of the following positions: position 73 where ARG is substituted with GLU; position 75 where ARG is substituted with ALA or GLU; position 76 where ARG is substituted with GLU; position 30 where ASP is substituted with ALA; position 56 where HIS is substituted with ALA; position 77 where TYR is substituted with PHE; position 88 where ARG is substituted with ALA; position 103 where ARG is substituted with ALA; position 109 where ASP is substituted with GLU; position 115 where ARG is substituted with ALA;
amino acid sequence of domain D1 of LAG-3 with a deletion of amino acid residues 54 to 66;
a recombinant soluble human LAG-3 Ig fusion protein (IMP321)—a 200-kDa dimer produced in Chinese hamster ovary cells transfected with a plasmid encoding for the extracellular domain of hLAG-3 fused to the human IgG1 Fc.

According to the invention there is also provided a pharmaceutical composition, which comprises (a) LAG-3 protein, or a derivative thereof that is able to bind to MHC class II molecules; (b) an anti-neoplastic agent, wherein the anti-neoplastic agent is a platinum-based anti-neoplastic agent or a topoisomerase I inhibitor; and (c) a pharmaceutically acceptable carrier, excipient, or diluent.

According to the invention there is further provided a combined preparation, or pharmaceutical composition, of the invention for use as a medicament.

The invention also provides a combined preparation, or pharmaceutical composition, of the invention for preventing, treating, or ameliorating cancer.

There is further provided according to the invention use of a combined preparation, or pharmaceutical composition, of the invention in the manufacture of a medicament for preventing, treating, or ameliorating cancer.

There is also provided according to the invention a method of preventing, treating, or ameliorating cancer, which comprises administering LAG-3 protein, or a derivative thereof that is able to bind to MHC class II molecules, and an anti-neoplastic agent, to a subject in need of such prevention, treatment, or amelioration, wherein the anti-neoplastic gent is a platinum-based anti-neoplastic agent or a topoisomerase I inhibitor.

The LAG-3 protein, or derivative thereof, and the anti-neoplastic agent may be administered sequentially to the subject, i.e. the LAG-3 protein, or derivative thereof, may be administered before, with, or after the anti-neoplastic agent.

The LAG-3 protein, or derivative thereof, and the anti-neoplastic agent may be administered to the subject within 96 hours, 72 hours, 48 hours, 24 hours, or 12 hours, of each other.

Alternatively, the LAG-3 protein, or derivative thereof, and the anti-neoplastic agent may be co-administered to the subject, for example as a composition comprising the LAG-3 protein, or derivative thereof, and the anti-neoplastic agent, or by simultaneous administration of separate doses of the LAG-3 protein, or derivative thereof, and the anti-neoplastic agent.

According to some embodiments, a plurality of doses of the LAG-3 protein, or derivative thereof, and/or a plurality of doses of the anti-neoplastic agent, is administered to the subject.

According to some embodiments, a dose of the LAG-3 protein, or derivative thereof, is administered before, with, or after each administration of two or more doses of the anti-neoplastic agent.

For example, a dose of the LAG-3 protein, or derivative thereof, may be administered within 96 hours, 72 hours, 48 hours, 24 hours, or 12 hours, of each administration of two or more doses of the anti-neoplastic agent.

The choice of appropriate dosages of the components used in combination therapy according to the present invention can be determined and optimized by the skilled person, for example, by observation of the patient, including the patient's overall health, and the response to the combination therapy. Optimization, for example, may be necessary if it is determined that a patient is not exhibiting the desired therapeutic effect or conversely, if the patient is experiencing undesirable or adverse side effects that are too many in number or are of a troublesome severity.

The doses of the components used in combination therapy according to the invention should be chosen to provide a therapeutically effective amount of the components in combination. An “effective amount” of the combination therapy is an amount that results in a reduction of at least one pathological parameter associated with cancer. For example, in some embodiments, an effective amount of the combination therapy is an amount that is effective to achieve a reduction of at least about 10%, 20%, 30%, 40%, 50%, 60%, 70%, 80%, or 90%, in the parameter, compared to the expected reduction in the parameter associated with the cancer without the combination therapy. For example, the parameter may be tumor growth.

According to the invention, combination treatment may be employed to increase the therapeutic effect of the anti-neoplastic agent, or LAG-3 protein, or derivative thereof, compared with the effect of the anti-neoplastic agent, or LAG-3 protein, or derivative thereof, as a monotherapy, or to decrease the doses of the individual components in the resulting combinations while preventing or further reducing the risk of unwanted or harmful side effects of the individual components.

In one embodiment, the LAG-3 protein, or derivative thereof, and the anti-neoplastic agent are each prescribed at a dose that is within a typically prescribed dose range for each compound as a monotherapy. The compounds may be prescribed as separate dosages or as a combination dosage. Such combinations provide increased efficacy compared with the effect of either compound as a monotherapy.

In another embodiment, the LAG-3 protein, or derivative thereof, and the anti-neoplastic agent are each prescribed at a dose that is below a typically prescribed dose for each component as a monotherapy, but at doses that have therapeutic efficacy in combination. The components may be prescribed as separate dosages or as a combination dosage. The dosages of the components in combination may be selected to provide a similar level of therapeutic efficacy as the LAG-3 protein, or derivative thereof, or the anti-neoplastic agent as a monotherapy, but with the advantage that the lower doses of the LAG-3 protein, or derivative thereof, and the anti-neoplastic agent reduce the risk of adverse side effects compared to the prescribed dosages of each compound as a monotherapy.

In another embodiment, the prescribed dosage of the anti-neoplastic agent is within a typically prescribed dose range for monotherapy, and the LAG-3 protein, or derivative thereof, is prescribed at a dosage that is below a typically prescribed dose for monotherapy.

In a further embodiment, the prescribed dosage of the anti-neoplastic agent is below a typically prescribed dose for monotherapy, and the LAG-3 protein, or derivative thereof, is prescribed at a dosage that is within a typically prescribed dose range for monotherapy.

Preferred dosages below the typically prescribed dose for monotherapy are doses that are up to 50%, or up to 25%, of the typically prescribed dose.

When administered in separate dosages, the LAG-3 protein, or derivative thereof, and the anti-neoplastic agent may be administered substantially simultaneously (for example, within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, about 72 hours, or about 96 hours, or more.

The skilled person will be able to determine, and optimise, a suitable time course for sequential administration, depending on the particular combination of the LAG-3 protein, or derivative thereof, and the anti-neoplastic agent. The time course is preferably selected such that there is at least one beneficial effect, for example an enhancing of the effect of the LAG-3 protein, or derivative thereof, or the anti-neoplastic agent, or a mutual enhancing of the effect of the combination components, for example a more than additive effect, additional advantageous effects, fewer side effects, less toxicity, or a combined therapeutic effect compared with a non-effective dosage of one or both of the combination components, and very preferably a synergism of the combination components.

It will be appreciated that the optimum time course will depend on the factors such as the time taken for the peak plasma concentration of the compound to be reached after administration, and the elimination half-life of each compound. Preferably the time difference is less than the half-life of the first component to be administered.

The skilled person will also be able to determine appropriate timing for administration. In certain embodiments, the anti-neoplastic agent may be administered in the morning, and the LAG-3 protein, or derivative thereof, administered at least once later in the day. In other embodiments, the anti-neoplastic agent and LAG-3 protein, or derivative thereof, may be administered at substantially the same time.

In some embodiments, the anti-neoplastic agent may be administered to the subject, for example, by a medical practitioner, and the subject may be provided with a dose of the

LAG-3 protein, or derivative thereof, for example in a pre-filled syringe, to administer later (for example later the same day, or the next day).

The subject may receive doses of the anti-neoplastic agent and LAG-3 protein, or derivative thereof, over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, or more.

Preferably the subject is a mammalian subject, more preferably a human subject.

Examples of cancers that may be treated according to the invention include breast, ovarian, lung, head, neck, endometrial, esophageal, bladder, cervical, osteogenic sarcoma, colon, colorectal cancer, lymphoma, and central nervous system or germ cell tumors.

In general, the components of a combination of the invention, or a composition of the invention, may be administered by known means, in any suitable formulation, by any suitable route. In some embodiments, the LAG-3 protein, or derivative thereof, is administered parenterally (including by subcutaneous, intravenous, or intramuscular injection). In some embodiments, the anti-neoplastic agent is administered intravenously. In particular embodiments, the LAG-3 protein, or derivative thereof, is administered subcutaneously, and the anti-neoplastic agent is administered intravenously.

Suitable pharmaceutical compositions and dosage forms may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate combinations or compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, for example, two tablets or capsules taken together may provide a therapeutically effective dosage, such that the unit dosage in each tablet or capsule is approximately 50% of the therapeutically effective amount.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

In addition to the formulations described previously, the active agent may be formulated as a depot preparation for controlled release of the active agent, preferably sustained release over an extended time period. These sustained release dosage forms are generally administered by implantation (for example, subcutaneously or intramuscularly or by intramuscular injection).

Combined preparations of the invention may be packaged with instructions for administration of the components on the combination. The instructions may be recorded on a suitable recording medium or substrate. For example, the instructions may be printed on a substrate, such as paper or plastic. The instructions may be present as a package insert, in the labeling of the container or components thereof (i.e., associated with the packaging or sub-packaging). In other embodiments, the instructions are present as an electronic storage data file present on a suitable computer readable storage medium, for example, CD-ROM, diskette. Some or all components of the combined preparation may be packaged in suitable packaging to maintain sterility.

Embodiments of the invention are described in the examples below, with reference to the accompanying drawings in which:

FIG. 1 shows the effect of administration of a LAG-3 derivative and Topotecan in the treatment of cancer;

FIG. 2 shows the effect of administration of a LAG-3 derivative and Carboplatin in the treatment of cancer;

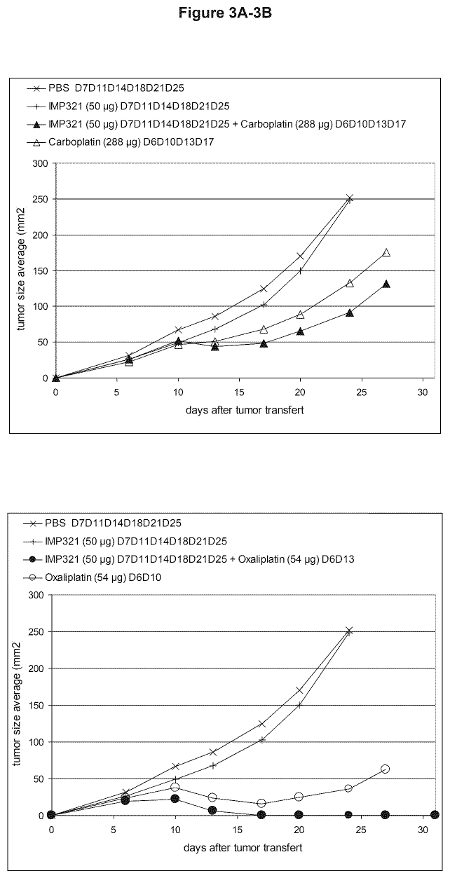

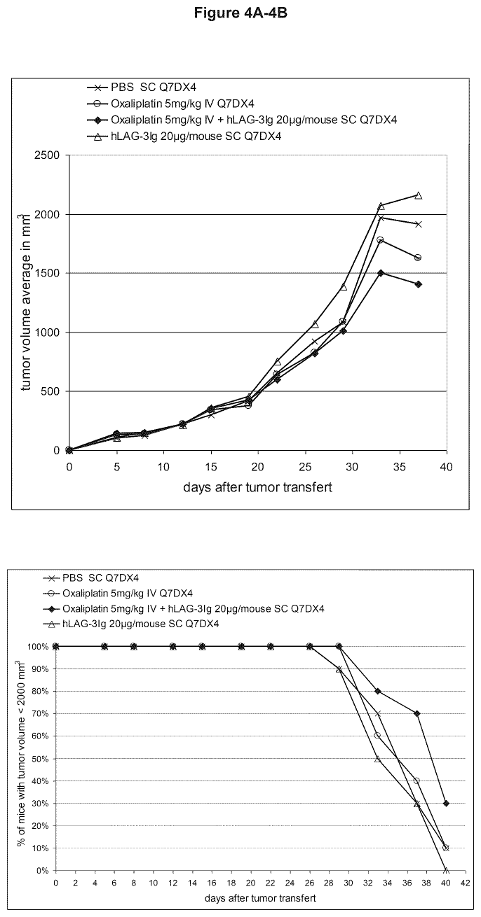

FIGS. 3A-3B shows the effect of administration of (A) a LAG-3 derivative and Carboplatin, or (B) a LAG-3 derivative and Oxaliplatin in the treatment of cancer;

FIGS. 4A-4B shows the effect of administration of a LAG-3 derivative and Oxaliplatin in the treatment of cancer: (A) shows the effect on tumor size, and (B) shows the effect on survival;

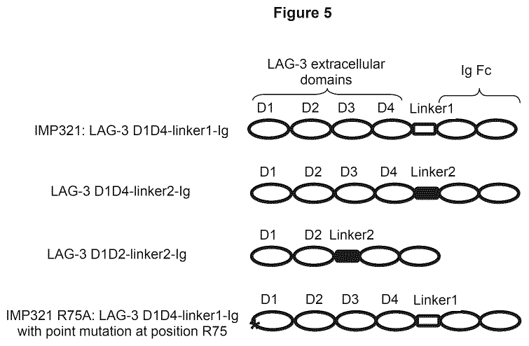

FIG. 5 shows an illustration of derivatives of LAG-3 protein fused to Immunoglobulin Fc (IgFc) sequence;

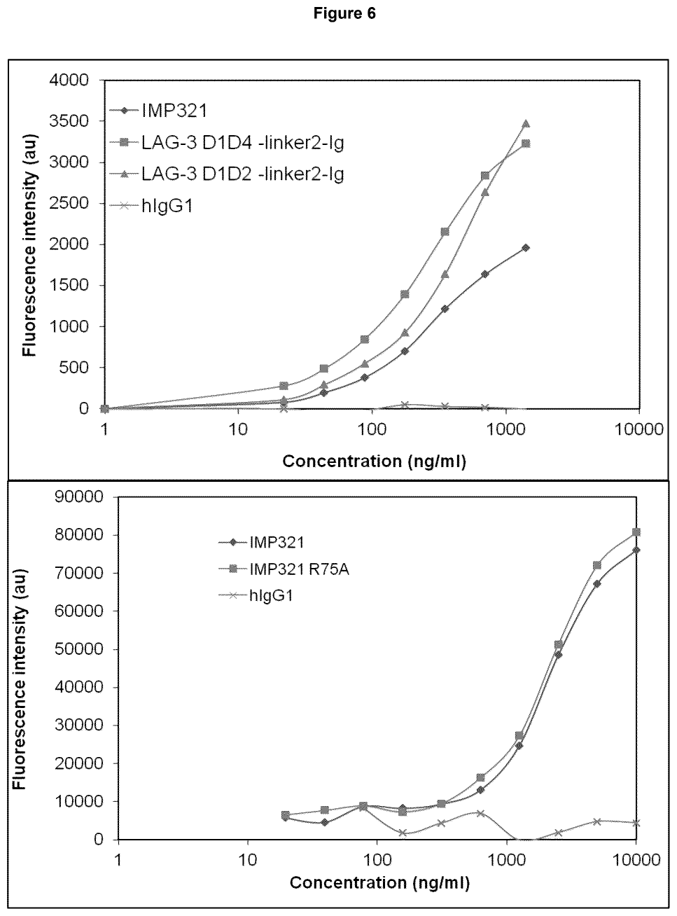

FIG. 6 shows binding of LAG-3 derivatives to MHC class II-positive cells;

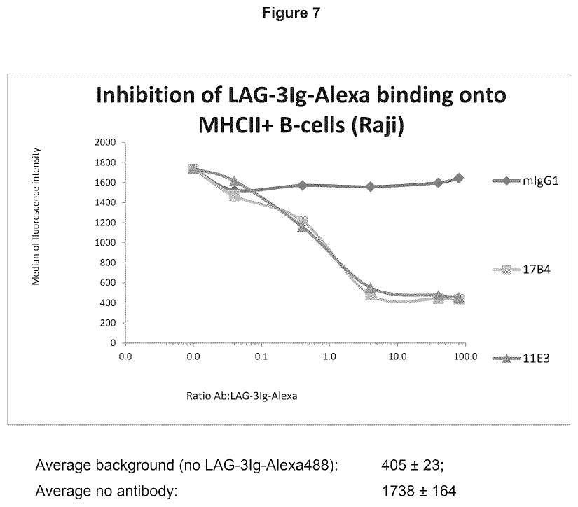

FIG. 7 shows inhibition of binding of a LAG-3 derivative to MHC class II-positive cells by antibodies that block binding of LAG-3 to MHC class II molecules;

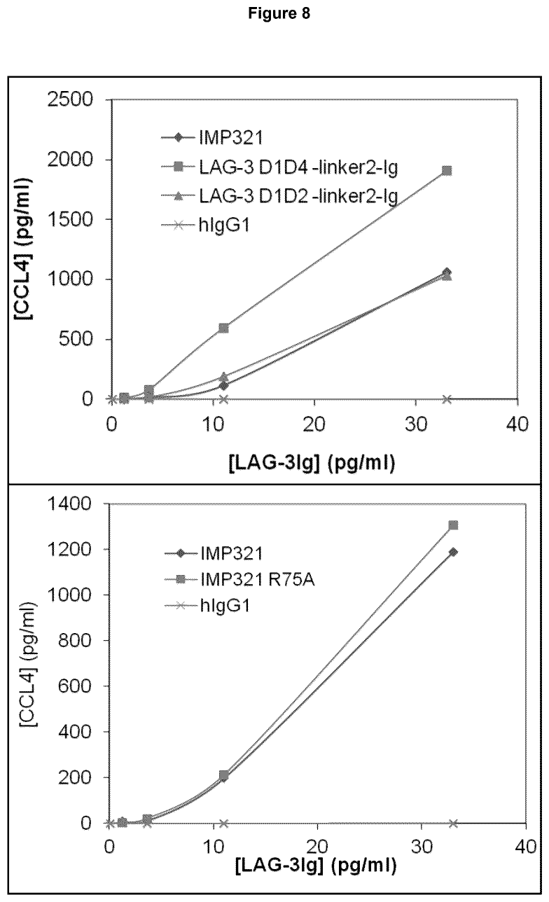

FIG. 8 shows activation of THP-1 cells by LAG-3 derivatives, as determined by CCL4 secretion;

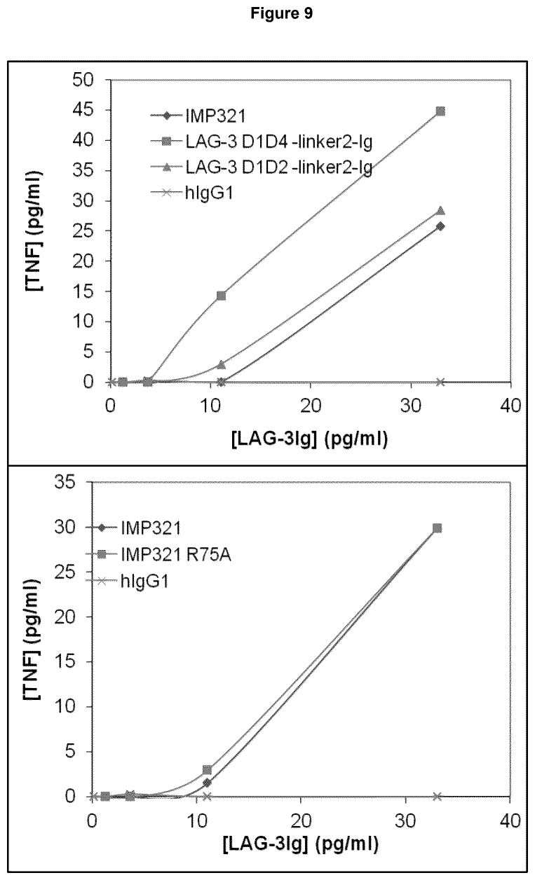

FIG. 9 shows activation of THP-1 cells by LAG-3 derivatives, as determined by TNF-α secretion;

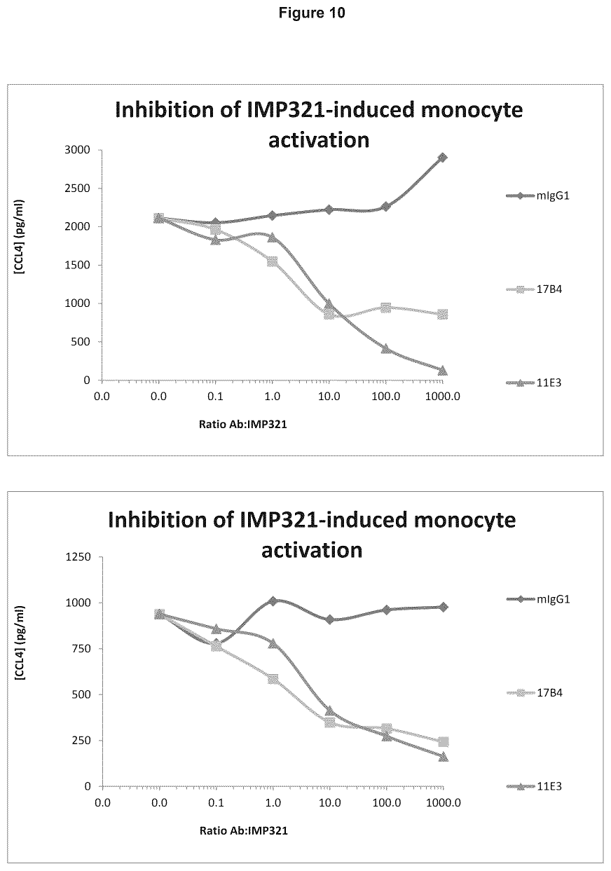

FIG. 10 shows inhibition of LAG derivative-induced monocyte activation by antibodies that block binding of LAG-3 to MHC class II molecules;

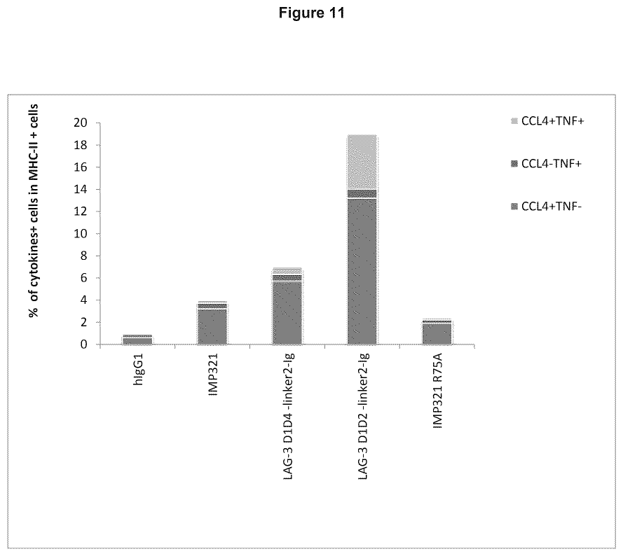

FIG. 11 shows activation of antigen-presenting cells (APCs) by LAG-3 derivatives;

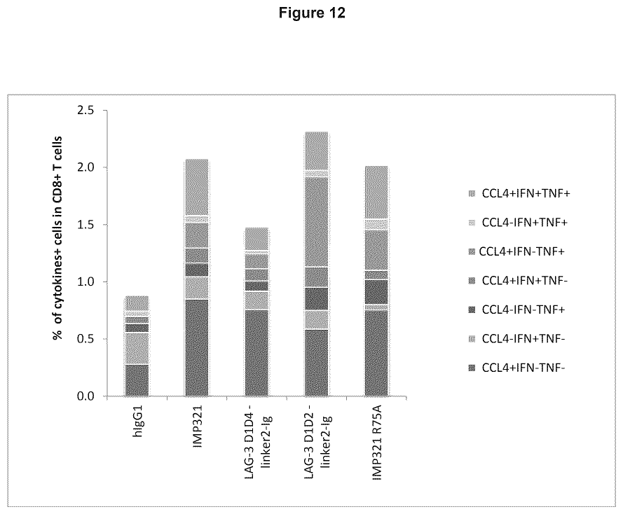

FIG. 12 shows activation of CD8-positive T cells by LAG-3 derivatives; and

FIG. 13 shows amino acid sequence of mature human LAG-3 protein. The four extracellular Ig superfamily domains are at amino acid residues: 1-149 (D1); 150-239 (D2); 240-330 (D3); and 331-412 (D4). The amino acid sequence of the extra-loop structure of the D1 domain of human LAG-3 protein is shown underlined in bold.

### EXAMPLE 1

### Effect of Administration of a LAG-3 Derivative and a Topoisomerase I Inhibitor in the Treatment of Cancer

A murine syngeneic skin tumour model was established using the colorectal adenocarcinoma cell line CT26.

One quarter of the minimum tumorigenic dose (MTD) of tumour cells (0.5×10⁵ cells) was implanted by subcutaneous (s.c.) injection in the right flank of four groups of BALB/c mice (5 weeks old) at Day 0. The mice were injected with phosphate buffered saline (PBS) (Group 1 mice), the LAG-3 derivative IMP321 (Group 2 mice), IMP321 and Topotecan (Group 3 mice), or Topotecan alone (Group 4 mice) according to the following schedule:
Group 1 (8 mice): negative control: PBS s.c. injection at D11, D14, D18, D21, D25 and D28;
Group 2 (7 mice): IMP321 s.c. injection (50 μg, 1.9 mg/ml) at D11, D14, D18, D21, D25 and D28;
Group 3 (8 mice): IMP321 s.c. injection (50 μg, 1.9 mg/ml) at D11, D14, D18, D21, D25 and D28, plus Topotecan i.p. injection (45 μg, 2.5 mg/kg) at D10, D13 and D17;
Group 4 (8 mice): Topotecan i.p. injection (45 μg, 2.5 mg/kg) at D10, D13 and D17.

Tumour growth was monitored twice a week by measuring two perpendicular tumour diameters using calipers. The results are shown in FIG. 1. Tumour size means the cross-sectional area in mm².

The results show that IMP321 alone had no effect on tumour growth, topotecan had some effect on reducing tumor growth, but combined treatment with IMP321 and topotecan has a greater (i.e. a synergistic) effect.

### EXAMPLE 2

Effect of Administration of a LAG-3 Derivative and a Platinum-Based Anti-Neoplastic Agent in the Treatment of Cancer

A murine syngeneic skin tumour model was established using the lymphoma cell line EL4.

The minimum tumorigenic dose (MTD) of tumour cells (5×10⁵ cells) was implanted by s.c. injection in the right flank of C57BI/6 mice (5 weeks old) at Day 0. The mice were injected with phosphate buffered saline (PBS) (Group 1 mice), IMP321 (Group 2 mice), IMP321 and Carboplatin (Group 3 mice), or Carboplatin alone (Group 4 mice) according to the following schedule:
Group 1 (8 mice): negative control: PBS s.c. injection at D7, D11, D14, D19, D21 and D24;
Group 2 (8 mice): IMP321 s.c. injection (50 μg, 3.96 mg/ml) at D7, D11, D14, D19, D21 and D24;
Group 3 (8 mice): IMP321 s.c. injection (50 μg, 3.96 mg/ml) at D7, D11, D14, D19, D21 and D24, plus Carboplatin i.p. injection (288 μg, 16 mg/kg) at D6, D10, D13 and D17;
Group 4 (8 mice): Carboplatin i.p. injection (288 μg, 16 mg/kg) at D6, D10, D13 and D17.

Tumour growth was monitored twice a week by measuring two perpendicular tumor diameters using calipers. The results are shown in FIG. 2. Tumour size means the cross-sectional area in mm².

The results show that IMP321 alone had no effect on tumour growth, carboplatin alone had very little effect, if any, but combined treatment with IMP321 and carboplatin reduced tumour growth, thereby demonstrating a synergistic effect of the combined administration.

### EXAMPLE 3

Effect of Administration of a LAG-3 Derivative and Different Platinum-Based Anti-Neoplastic Agents in the Treatment of Cancer

A murine syngeneic skin tumour model was established using the lymphoma cell line EL4.

The minimum tumorigenic dose (MTD) of tumour cells (5×10⁵ cells) was implanted by s.c. injection in the right flank of C57BI/6 mice (5 weeks old) at Day 0. The mice were injected with phosphate buffered saline (PBS) (Group 1 mice), IMP321 (Group 2 mice), IMP321 and Carboplatin (Group 3 mice), Carboplatin alone (Group 4 mice), IMP321 and Oxaliplatin (Group 5 mice), or Oxaliplatin alone (Group 6 mice) according to the following schedule:
Group 1 (10 mice): negative control: PBS s.c. injection at D7, D11, D14, D18, D21 and D25;
Group 2 (10 mice): IMP321 s.c. injection (50 μg, 1.9 mg/ml) at D7, D11, D14, D18, D21 and D25;
Group 3 (9 mice): IMP321 s.c. injection (50 μg, 1.9 mg/ml) at D7, D11, D14, D18, D21 and D25, plus Carboplatin i.p. injection (288 μg, 16 mg/kg) at D6, D10, D13 and D17;
Group 4 (10 mice): Carboplatin i.p. injection (288 μg, 16 mg/kg) at D6, D10, D13 and D17;
Group 5 (10 mice): IMP321 s.c. injection (50 μg, 1.9 mg/ml) at D7, D11, D14, D18, D21 and D25, plus Oxaliplatin i.p. injection (54 μg, 3 mg/kg) at D6 and D10;
Group 6 (10 mice): Oxaliplatin i.p. injection (54 μg, 3 mg/kg) at D6 and D10.

Tumour growth was monitored twice a week by measuring two perpendicular tumor diameters using calipers. The results are shown in FIG. 3A (for Carboplatin) and FIG. 3B (for Oxaliplatin). Tumour size means the cross-sectional area in mm².

The results show that IMP321 alone had little, if any effect, carboplatin alone had some effect, and combined treatment with IMP321 and carboplatin had a greater (i.e. a synergistic) effect. Oxaliplatin alone had an effect, but combined treatment with IMP321 and oxaliplatin had an even greater (i.e. a synergistic) effect, with tumour growth completely inhibited by Day 17.

### EXAMPLE 4

Effect of administration of a LAG-3 derivative and a platinum-based anti-neoplastic agent in the treatment of cancer

The effect of combined treatment with the LAG-3 derivative IMP321 (also referred to as hLAG-3 Ig) and oxaliplatin in the C38 colon adenocarcinoma tumor model was evaluated. In this model, tumor fragments are surgically implanted subcutaneously. When treatment is begun (at day 12, when the mean tumor volume is 200 mm³), the tumor is relatively mature and so provides a good model for real-life tumors.

Mouse colon 38 (C38) tumor fragments were obtained frozen from the Division of Cancer Treatment, Tumor Repository, NCI (Frederick, MD, USA). The C38 fragments were stored frozen in DMSO/SVF/RPMI 1640 medium (10/10/80) in liquid nitrogen until use. The fragments were thawed at 37° C. for 5 min, rinsed twice in RPMI 1640 medium before subcutaneous (SC) implantation in mice. The C38 tumors were serially transplanted in C57BI/6 mice.

Small C38 tumor fragments (20-30 mg) were subcutaneously implanted in the right flank of 12 C57BL/6 mice. When tumor sizes reached 500-1000 mm³, tumors were surgically excised and small C38 tumor fragments (20-30 mg) were subcutaneously implanted in the right flank of recipient C57BL/6 mice.

Treatment started when the tumors reached a mean volume of 200-300 mm³. The treatment schedule was as follows:
Group 1 (10 mice): one weekly SC injection of PBS for 4 consecutive weeks;
Group 2 (10 mice): one weekly IV injection of oxaliplatin at 5 mg/kg/inj for 4 consecutive weeks;
Group 3 (10 mice): one weekly SC injection of 20 μg IMP321 for 4 consecutive weeks;
Group 4 (10 mice): one weekly IV injection of oxaliplatin at 5 mg/kg/inj in combination with one weekly SC injection of 20 μg IMP321 for 4 consecutive weeks.

Treatment started at day 12 (D12) when the different groups had a mean tumor volume of 200 mm³. PBS or Oxaliplatin was injected at D12, D19, D26 and D33. IMP321 was injected the day after Oxaliplatin, that is at D13, D20, 27 and D34. Animals were terminated when the subcutaneous tumor reached a maximum volume of 2,000 mm³:

| | | | Administration | Treatment |
|---|---|---|---|---|
| Group | Treatment | Dose | route | schedule |
| 1 | PBS | — | SC | Q7Dx4 |
| 2 | Oxaliplatin | 5 mg/kg/inj | IV | Q7Dx4 |
| 3 | IMP321 | 20 μg/mouse/inj | SC | Q7Dx4* |
| 4 | Oxaliplatin | 5 mg/kg/inj | IV | Q7Dx4 |
| | IMP321 | 20 μg/mouse/inj | SC | Q7Dx4* |

*performed the day after the treatment with oxaliplatin

The results are shown in FIG. 4A. The results show that IMP321 alone had no effect on delaying tumor growth. Oxaliplatin had a slight effect. The combination of Oxaliplatin and IMP321 had a greater effect. The same synergistic effect is seen in the survival curves, shown in FIG. 4B.

### EXAMPLE 5

Binding of LAG-3 derivatives to MHC class II-positive cells

Several derivatives of LAG-3 were tested for their ability to bind to MHC class II-positive cells:
i) domains D1-D4 of LAG-3 linked to immunoglobulin Fc (Ig Fc) sequence by a first linker (LAG-3 D1D4-linker1-Ig, sLAG-3 D1D4-Ig, or IMP321);
ii) domains D1-D4 of LAG-3, linked to Ig Fc sequence by a second linker (LAG-3 D1D4-linker2-Ig, or sLAG-3 D1D4-linkerB-Ig);
iii) domains D1 and D2 of LAG-3, linked to Ig Fc sequence by the second linker (LAG-3 D1D2-linker2-Ig, or sLAG-3 D1D2-linkerB-Ig); and
iv) domains D1-D4 of LAG-3 linked to Ig Fc sequence by the first linker, but with a mutation in the MHC class II binding site of the D1 domain of LAG-3, at position R75 (R75A), which enhances binding to MHC class II molecules by three-fold or more (Huard et al., Proc. Natl. Acad. Sci. USA, 1997, 94:5744) (IMP321 R75A).

The derivatives are illustrated in FIG. 5.

MHC class II+ Raji cells were incubated for 45 minutes at 4° C. with various concentrations of the LAG-3 derivatives, or with a human IgG1 antibody (hIgG1) as a negative control. The LAG-3 molecules bound to the cell surface were revealed with a FITC-conjugated goat anti-mouse Ig (Coulter). The cells were analyzed by flow cytometry. The results, expressed as fluorescence intensity units, are shown in FIG. 6. The results show that all of the LAG-3 derivatives bound to MHC class II-positive cells.

### EXAMPLE 6

Inhibition of binding of the LAG-3 derivative IMP321 to MHC class II-positive cells by antibodies that block binding of LAG-3 to MHC class II molecules 17B4 and 11E3 are anti-LAG-3 monoclonal antibodies that are known to block binding of LAG-3 to MHC class II molecules. Binding of an IMP321-label conjugate (LAG-3 Ig-Alexa 488) to MHC class II-positive B cells (Raji cells) was determined following pre-incubation of the conjugate (4 μg/ml at 4° C.) with 17B4 or 11E3 blocking antibody, or with an isotype-matched negative control monoclonal antibody (mIgG1). Analysis of cell-bound fluorescence was carried out using fluorescence-activated cell sorting (FACS). The results are shown in FIG. 7.

The results show that binding of IMP321 to Raji cells was inhibited by LAG-3-specific monoclonal antibody that blocks binding of LAG-3 to MHC class Il molecules.

### EXAMPLE 7

### Activation of Monocytes by LAG-3 Derivatives

THP-1 cells were incubated for 4 hours at 4° C. with the LAG-3 derivatives illustrated in FIG. 5, or with human IgG1 as a negative control. The amount of secretion by the THP-1 cells of the chemokine CCL4, and the cytokine Tumor Necrosis Factor-α, TNF-α, was determined, and was used as a measure of monocyte activation. CCL4 and TNF-α secretion was quantified in the cell supernatants using a Cytometric Beads Array. The results of the CCL4 determinations are shown in FIG. 8, and the results of the TNF-α determinations are shown in FIG. 9.

The results show that the LAG-3 derivates were all able to activate THP-1 monocytic cells.

### EXAMPLE 8

Inhibition of IMP321-Induced Monocyte Activation by Antibodies that Block Binding of LAG-3 to MHC Class II Molecules

IMP321 (20 ng/ml) was preincubated with 17B4 or 11E3 antibody (at +37° C.), before incubation of the mixture with THP-1 cells for 4 hours. The amount of CCL4 secretion by the THP-1 cells was used to determine the level of monocyte activation. The results of two experiments are shown in FIG. 10.

The results demonstrate that IMP321-induced monocyte activation is inhibited by the blocking anti-LAG-3 mAbs 17B4 and 11E3. This indicates that the ability of IMP321 to activate monocytes is dependent on binding of IMP321 to MHC class Il molecules.

### EXAMPLE 9

Activation of primary antigen-presenting cells (APCs) by LAG-3 derivatives

Human peripheral blood mononuclear cells (PBMCs) were incubated for 4 hours with the LAG-3 derivatives illustrated in FIG. 5, or with human IgG1 as a negative control, in the presence of brefeldin, a secretion inhibitor. The cytokine response of the APCs present in the PBMCs was determined by intracellular staining of CCL4, a chemokine known to favour the Th1 and CD8-positive response, and TNF-γ multifunctional cytokine which directly inhibits tumorigenesis. The results were analyzed by cytometry. The results, represented by the percentage of cells expressing CCL4 and/or TNF-α in MHC class II-positive cells, are shown in FIG. 11.

The results show that all the LAG-3 derivatives tested induced the production of CCL4, and TNF-α.

### EXAMPLE 10

Activation of T Cells by LAG-3 Derivatives

Human PBMCs were incubated for 18 hours with the LAG-3 derivatives illustrated in FIG. 5, or with human IgG1 as a negative control. Brefeldin was present for the last 16hours of the incubation. The cytokine response of CD8-positive T cells after 18 hour exposure to LAG-3 derivatives was followed by intracellular staining of CCL4, IFN-γ and TNF-α and analyzed by cytometry. The results, represented as the percentage of cells expressing CCL4, IFN-γ and/or TNF-α in CD3-positive/CD8-positive T cells, are shown in FIG. 12.

The results show that all of the LAG-3 derivatives tested induced activation of Type 1 cytotoxic CD8-positive T cells (Tc1 cells). It is concluded that, through binding to MHC class II molecules expressed by APCs, the LAG-3 derivatives induced activation of Tc1 cells. Activation of Tc1 cells forms the main anti-tumor immune response.

1. A combined preparation, which comprises: (a) LAG-3 protein, or a derivative thereof that is able to bind to MHC class II molecules; and (b) an anti-neoplastic agent, wherein the anti-neoplastic agent is a platinum-based anti-neoplastic agent or a topoisomerase I inhibitor.

2. A combined preparation according to claim 1, for co-administration or sequential administration of the LAG-3 protein, or derivative thereof, and the anti-neoplastic agent.

3. A combined preparation according to claim 1 or 2, wherein the LAG-3 protein, or derivative thereof, is separate from the anti-neoplastic agent.

4. A combined preparation according to any preceding claim, wherein the LAG-3 protein, or derivative thereof, is present at a dose which is a molar equivalent of 0.25-30 mg of LAG-3 Ig fusion protein IMP321.

5. A combined preparation according to any preceding claim, which comprises a plurality of doses of the LAG-3 protein, or derivative thereof.

6. A combined preparation according to any preceding claim, which comprises a plurality of doses of the anti-neoplastic agent.

7. A combined preparation according to any preceding claim, wherein the platinum-based anti-neoplastic agent comprises oxaliplatin or carboplatin.

8. A combined preparation according to any of claims 1 to 6, wherein the topoisomerase I inhibitor comprises topotecan.

9. A combined preparation according to any preceding claim, wherein the derivative of LAG-3 protein comprises an amino acid sequence that has at least 70% amino acid identity with domain D1, and optionally domain D2, of LAG-3 protein, preferably human LAG-3 protein.

10. A combined preparation according to any preceding claim, wherein the derivative of LAG-3 protein comprises an amino acid sequence that has at least 70% amino acid identity with domains D1, D2, D3, and optionally D4, of LAG-3 protein, preferably human LAG-3 protein.

11. A combined preparation according to any preceding claim, wherein the derivative of LAG-3 protein is fused to Immunoglobulin Fc sequence.

12. A pharmaceutical composition, which comprises (a) LAG-3 protein, or a derivative thereof that is able to bind to MHC class II molecules; (b) an anti-neoplastic agent, wherein the anti-neoplastic agent is a platinum-based anti-neoplastic agent or a topoisomerase I inhibitor; and (c) a pharmaceutically acceptable carrier, excipient, or diluent.

13. A combined preparation according to any of claims 1 to 11, or a pharmaceutical composition according to claim 12, for use as a medicament.

14. A combined preparation according to any of claims 1 to 11, or a pharmaceutical composition according to claim 12, for preventing, treating, or ameliorating cancer.

15. Use of a combined preparation according to any of claims 1 to 11, or a pharmaceutical composition according to claim 12, in the manufacture of a medicament for preventing, treating, or ameliorating cancer.

16. A method of preventing, treating, or ameliorating cancer, which comprises administering LAG-3 protein, or a derivative thereof that is able to bind to MHC class II molecules, and an anti-neoplastic agent, to a subject in need of such prevention, treatment, or amelioration, wherein the anti-neoplastic agent is a platinum-based anti-neoplastic agent or a topoisomerase I inhibitor.

17. A method according to claim 16, wherein the LAG-3 protein, or derivative thereof, and the anti-neoplastic agent are administered sequentially to the subject.

18. A method according to claim 16, wherein the LAG-3 protein, or derivative thereof, is administered after the anti-neoplastic agent.

19. A method according to claim 17 or 18, wherein the LAG-3 protein, or derivative thereof, and the anti-neoplastic agent are administered to the subject within 96 hours of each other.

20. A method according to claim 16, wherein the LAG-3 protein, or derivative thereof, and the anti-neoplastic agent are co-administered to the subject.

21. A method according to any of claims 16 to 20, wherein the LAG-3 protein, or derivative thereof, is administered to the subject at a dose which is a molar equivalent of 0.25-30 mg of LAG-3 Ig fusion protein IMP321.

22. A method according to any of claims 16 to 21, wherein a plurality of doses of the LAG-3 protein, or derivative thereof, is administered to the subject.

23. A method according to any of claims 16 to 22, wherein a plurality of doses of the anti-neoplastic agent is administered to the subject.

24. A method according to claim 22 or 23, wherein a dose of the LAG-3 protein, or derivative thereof, is administered before, with, or after each administration of two or more doses of the anti-neoplastic agent.

25. A method according to any of claims 16 to 24, wherein the platinum-based anti-neoplastic agent comprises oxaliplatin or carboplatin.

26. A method according to any of claims 16 to 25, wherein the topoisomerase I inhibitor comprises topotecan.

27. A method according to any of claims 16 to 26, wherein the derivative of LAG-3protein comprises an amino acid sequence that has at least 70% amino acid identity with domain D1, and optionally domain D2, of LAG-3 protein, preferably human LAG-3 protein.

28. A method according to any of claims 16 to 27, wherein the derivative of LAG-3 protein comprises an amino acid sequence that has at least 70% amino acid identity with domains D1, D2, D3, and optionally D4, of LAG-3 protein, preferably human LAG-3 protein.

29. A method according to any of claims 16 to 28, wherein the derivative of LAG-3 protein is fused to Immunoglobulin Fc sequence.
